(19) United States
(12) Patent Application Publication
(10) Pub. No.: US 2023/0120931 A1
(43) Pub. Date: Apr. 20, 2023

(54) METHOD FOR LOCALIZING SINGLE MOLECULES OF A DYE IN A SAMPLE AND FOR GENERATING HIGH-RESOLUTION IMAGES OF STRUCTURE IN A SAMPLE

(71) Applicant: Abberior Instruments GmbH, Gottingen (DE)

(72) Inventor: Lars KASTRUP, Gottingen (DE)

(21) Appl. No.: 18/077,817

(22) Filed: Dec. 8, 2022

Related U.S. Application Data
(63) Continuation-in-part of application No. PCT/EP2021/067068, filed on Jun. 23, 2021.

(30) Foreign Application Priority Data
Jun. 23, 2020 (DE) 10 2020 116 547.1

(51) Int. Cl. G01N 21/64; G01N 33/58

(52) U.S. Cl. CPC G01N 21/6458; G01N 21/6428; G01N 33/582; G01N 2021/6439

(57) ABSTRACT

The present disclosure relates to methods for generating high-resolution images of a structure in a sample or for localizing individual molecules of a fluorescent dye in a sample, and the use of a fluorescent dye in such a method. The methods according to the present disclosure are characterized in that the fluorescent dye is first formed from a protected, non-fluorescent form of the dye in a photoactivation reaction comprising at least two reaction steps prior to scanning with excitation and fluorescence inhibition light, and that the protected, non-fluorescent form of the dye is inert to the excitation and fluorescence inhibition light.

### CROSS-REFERENCE TO RELATED APPLICATIONS

The present application is a continuation-in-part application and claims priority to and the benefit of International Patent Application No. PCT/EP2021/067068, filed on Jun. 23, 2021, the entire contents of which is incorporated herein by reference.

### TECHNICAL FIELD OF THE PRESENT DISCLOSURE

The present disclosure relates to methods for generating high-resolution images of a structure or for localizing individual molecules of a fluorescent dye in a sample. For this purpose, the sample or a section of the sample is scanned with excitation light and an intensity distribution of fluorescence inhibition light comprising a local minimum, which inhibits, reduces or completely suppresses the emission of fluorescent light by the fluorescent dye. Compared to related methods known from the prior art, the method according to the present disclosure is characterized in that prior to scanning with excitation and fluorescence inhibition light the fluorophore is first formed from a protected, non-fluorescent form of the dye in a photoactivation reaction comprising at least two reaction steps inhibition light, and that the protected, non-fluorescent form of the dye is inert to the excitation and fluorescence inhibition light.

### PRIOR ART

STED microscopy is a laser scanning microscopy technique that allows high spatial resolution imaging of a sample labeled with a fluorescent dye. In this process, the sample is scanned with focused excitation light and with stimulation light, wherein the intensity distributions of the excitation and stimulation light are largely complementary and the intensity distribution of the stimulation light comprises a zero at the location of the intensity maximum of the excitation light. At high intensity locations, the stimulation light inhibits the fluorescent dye from emitting fluorescence, thereby limiting fluorescence emission to a small region around the zero of the intensity distribution of the stimulation light and reducing the magnitude of the effective point spread function.

As a further development of STED microscopy, DE 10 2013 100 172 A1 discloses a method for STED microscopy in which the sample in the measurement area is additionally illuminated with an intensity distribution of fluorescence inhibition light—which, like the stimulation light, has a local minimum—before being illuminated with excitation light and stimulation light, which converts the fluorophore from the fluorescent state into a protective state in which the fluorophore is protected from electronic excitation by the excitation light and the stimulation light. In this context, the aim of the method is not primarily to improve the resolving power of the microscope by superimposing the intensity distributions of the stimulation light and the fluorescence inhibition light; rather, the photobleaching of the fluorophore by the excitation light and the stimulation light is to be reduced, particularly in areas of high intensity of the stimulation light, by transferring the fluorophore, particularly in these areas, to the non-excitable protective state and thus protecting it from the bleaching effect of the excitation light and stimulation light. A prerequisite for the application of the method is the availability of suitable fluorophores that can be temporarily transferred to the protective state, i.e., that can be photo switched. The requirements for the switching contrast are less stringent than, for example, for RESOLFT microscopy but the protected state must be (largely) inert to the excitation and stimulation light—a requirement that is not or only insufficiently fulfilled in practice for many photo switchable fluorophores.

Other related methods for high-resolution fluorescence microscopy are known under the collective term localization microscopy. These methods are based on the highly accurate spatial localization of single fluorescent dye molecules and the reconstruction of a high-resolution image from these single-molecule localizations. A prerequisite for the application of these methods is that fluorescent dye molecules are present in the sample at each time point in a singulated manner, i.e., spatially separated from each other, so that they appear as isolated objects in the camera image when imaged onto a camera. Spatially separated fluorescent dye molecules can be achieved, e.g., by using an appropriately high dilution of the dye when labeling the sample; however, in order to be able to generate a high-resolution image of the structure that is as spatially continuous as possible, it is necessary to label the structure with many fluorescent dye molecules in high density and to determine the locations of a sufficiently large share of these fluorescent dye molecules, typically several thousand fluorescent dye molecules. The high labeling density implies that during a localization of dye molecules, only a small fraction of all dye molecules may be present in a fluorescent state at any given time in order to meet the requirement that fluorescent molecules be present individually and spatially isolated. In localization microscopy, one therefore uses photo switchable fluorescent dyes which have a fluorescent state in which the dye can be excited to fluorescence with excitation light of suitable wavelength, and which furthermore have a dark state in which the dye cannot be excited to fluorescence with the excitation light. In this case, the dye can be photoactivated at least once, i.e., converted from the dark state to the fluorescent state. Photoactivation is often light-induced, i.e., by illumination with photoactivation light of suitable wavelength (usually in the blue-violet spectral range), which allows the proportion of photoactivated dye molecules to be precisely adjusted and controlled. Alternatively, dye molecules can spontaneously switch to an activated state. Depending on the type of fluorescent dye, activation can also be reversible, i.e., multiple activation-deactivation switching cycles can be undergone, and deactivation or transition to a non-fluorescent state can also be light-induced or spontaneous. A typical switching mechanism is the (reversible) transition from the fluorescent state to a transient dark state, such as a triplet state. The switching kinetics of these transitions can be adapted to the respective requirements, among other things, via the solvent composition, by adding redox reagents and/or by controlling the oxygen concentration in the sample.

The localization accuracy achievable in localization microscopy depends on several factors and was reported, for example, by R. E. Thompson et al, Biophys. J. 82, 2775 (2002) as:

$$σ = \sqrt{\frac{{s}^{2}}{N} + \frac{{a}^{2}}{12 N} + \frac{4 \sqrt{π} {s}^{3} {b}^{2}}{{aN}^{2}}} , \qquad ( 1 )$$

wherein s is the width (standard deviation) of a PSF of the microscope approximated as a Gaussian function, N is the number of detected fluorescence photons of the dye molecule, a is the edge length of the detector pixels and b is the background signal composed of background fluorescence and detector noise.

Provided that the localization accuracy is not significantly affected by the size of the detector pixels a or by the background signal b equation (1) shows that with an (effective) PSF of smaller width s at a given photon number N a higher localization accuracy resp. a smaller localization uncertainty σ or a desired localization accuracy can be achieved with a smaller number of photons N. A narrower effective PSF (with a smaller value s) can be generated, e.g., by not illuminating the sample with homogeneously distributed excitation light over a large area but, in analogy to STED microscopy, with focused excitation and stimulation light that scans the image field by means of a scanning device. A Gaussian excitation focus is overlaid with an intensity distribution of stimulation light with a local intensity minimum, which suppresses fluorescence emission in the edge regions of the excitation focus and thus reduces the width s of the effective PSF. Since scanning illumination of the entire image field is inherently slower than wide-field illumination, the imaging scheme may need to be adapted so that the image field is scanned in sections rather than as a whole, with the sections being selected, e.g., to each contain a fluorescent dye molecule.

More recently, features of the above techniques were combined in MINFLUX nanoscopy, first described by F. Balzarotti et al. in arXiv:1611.03401 [physics.optics]. In this method, spatially isolated fluorescent molecules are illuminated with an intensity distribution comprising an intensity minimum of excitation light at a sequence of different positions. For each of the illumination positions, the fluorescence emission excited by the excitation light is registered, and the position of the fluorescent molecule is inferred from the set of registered values of the intensity of the fluorescent light. By its nature, this position determination is subject to uncertainty; however, the uncertainty of the position determination can be reduced by applying the method iteratively. For this purpose, the illumination positions are adjusted before each iteration step, i.e., arranged closer around the respective assumed position of the molecule. At the same time, the strength of the excitation light is increased so that the intensity gradient increases near the intensity minimum. Alternatively, the measurement time can be increased, which corresponds to an increase in the strength of the excitation light with respect to the amount of effective light. With the parameters adjusted, the molecule is successively illuminated at each of the adjusted illumination positions and the intensity of the fluorescence emission is recorded. From the dependence of the fluorescence signal on the positions of the intensity minimum, the position of the molecule can now be determined with less uncertainty than before. These method steps can be repeated until the position determination has converged or until another termination criterion is reached, e.g., a predetermined maximum acceptable uncertainty. With an achievable localization accuracy of about 1 nm, the MINFLUX method represents the most precise commercially available localization method for fluorescent molecules according to the current prior art.

WO 2015/097000 A1 further discloses that a (high-resolution) image of the distribution of the molecules in the sample can be obtained from the position data of the individual molecules (“MINFLUX imaging”). This method corresponds to the procedures generally known from localization microscopy for generating high-resolution images from a large number of position determinations of individual fluorescent molecules, but in the case of MINFLUX nanoscopy results in a further increased spatial resolution of the images of 5 nm or better.

DE 10 2017 104 736 B3 describes a modification of the MINFLUX method in which the scanning of the isolated fluorescent dye molecules is not performed by illuminating them with an intensity distribution of excitation light having a local intensity minimum, but with two essentially complementary intensity distributions of excitation and fluorescence inhibition light. In this case, the intensity distribution of the excitation light comprises a local intensity maximum, while the intensity distribution of the fluorescence inhibition light comprises a local intensity minimum at the same location. Specifically, the fluorescence inhibition light may be stimulation light that prevents excited fluorescent dye molecules from emitting fluorescence photons in the edge regions of the intensity distribution of the excitation light by triggering stimulated emission. Thus, the excitation light and the fluorescence inhibition light are superimposed with such intensity distributions as is done in RESOLFT and STED microscopy. It is exploited that the intensity of the fluorescence light registered for the particular fluorescent dye molecule depends on its distance from the local intensity minimum of the fluorescence inhibition light, and that its position can be determined with high accuracy from the intensities of the fluorescence light registered for several positions of the intensity minimum of the fluorescence inhibition light. Also with this modification of the MINFLUX method, the local intensity minimum can be positioned at a few positions in the sample, and the evaluation of the intensities of the registered fluorescent light can be performed according to the same principles as in MINFLUX nanoscopy. As a difference, however, it remains that in MINFLUX nanoscopy the intensity of the fluorescence light from the fluorescence marker increases with increasing distance of its position from the position of the local intensity minimum, while in the modification of the method in which the further light is fluorescence inhibition light, it decreases with increasing distance.

In summary, several variants of high-resolution fluorescence microscopy are known from the prior art, in which stimulation light is used in addition to excitation light to achieve high spatial resolution in the image. At the same time, these techniques require the use of photoactivatable or photoswitchable fluorescent dyes to label the sample. In practice, the applicability of these methods is limited because the commonly used photoactivatable fluorescent dyes with a photolabile protective group are not or not sufficiently inert, especially to illumination with high intensity stimulation light. This is particularly true if the stimulation light is used in the form of short laser pulses with very high peak intensities—as is preferred in STED microscopy. In this case, multiphoton absorption by the photolabile protective groups is also increased, resulting in undesirable photoactivation of the fluorescent dye.

### OBJECTIVE OF THE PRESENT DISCLOSURE

It is therefore the object of the present disclosure to provide methods for high-resolution fluorescence microscopy in which photoactivatable fluorescent dyes can be exposed to high intensities of stimulation light or other fluorescence inhibition light without the photoactivatable fluorescent dyes being activated by the stimulation light or fluorescence inhibition light in a way disturbing the method but rather is inert to that light.

### Solution

The objective of the present disclosure is attained by methods according to the independent claims. The dependent claims relate to preferred embodiments of the method. A Use claim is directed to the use of a photoactivatable fluorescent dye in a method according to the present disclosure.

The methods according to the present disclosure are based on the methods of high-resolution fluorescence microscopy known from the prior art, in which fluorescence inhibition or stimulation light is used in addition to excitation light to achieve a high spatial resolution in the image. However, they improve the known methods in a decisive way in that a type of photoactivation of fluorescent dyes is used that can greatly reduce or completely suppress unintentional and disturbing photoactivation by stimulation or fluorescence inhibition light. This greatly extends the range of application compared with known methods or makes it possible to use the method at all.

The present disclosure is based on the applicant's insight that, while protected non-fluorescent fluorescent dyes typically have a small absorption cross-section at the wavelength of the stimulation light, this small absorption cross-section is still sufficient to lead to undesirable photoactivation given the very high intensities of the fluorescence inhibition or stimulation light used oftentimes. In addition, two-photon or multiphoton absorption of the fluorescence inhibition or stimulation light may occur in some circumstances, which can also lead to photoactivation. Some of the photolabile protecting groups used to prepare photoactivatable fluorescent dyes are even optimized for multiphoton absorption.

The stability of the photoactivatable fluorescent dyes to fluorescence inhibition or stimulation light can now be significantly improved by designing the photoactivation of the protected, non-fluorescent dye in such a way that activation occurs in several reaction steps rather than in one. In this case, if each of the reaction steps is light-induced and the fluorescence capability is established only after the last reaction step, photoactivation therefore requires the absorption of two (or more) photons, resulting in a nonlinear (i.e., quadratic, cubic, . . . ) dependence of the photoactivation rate on light intensity analogous to two-photon/multiphoton fluorescence. As known from two-photon fluorescence excitation, the phenomenon of two-photon absorption is significant in practice only when very short light pulses are used, which can equally be applied to photoactivation during the transition from single-photon to two-photon absorption. Undesired photoactivation can thus be lowered, which, by the way, also applies in the case of multiphoton absorptions, which become absorption processes of (even) higher order.

Although the probability of photoactivation is also reduced with dedicated activation light, this is usually available in sufficient intensity and can in particular be dosed independently of the stimulation light. Due to the higher order of the photoactivation process, photoactivation remains confined to a smaller volume and therefore allows even more spatially precise control of photoactivation.

### DESCRIPTION OF THE PRESENT DISCLOSURE

The present disclosure relates to three methods, linked by one and the same inventive concept, for producing high-resolution images of a structure in a sample or for localizing individual molecules of a fluorescent dye in a sample, and to the use of a fluorescent dye in such a method. Common to all three methods is that a photoactivatable fluorescent dye is selected in which the photoactivation comprises at least two respective light-induced reaction steps. Further, in all methods according to the present disclosure, photoactivation of the fluorescent dye occurs in an activation reaction comprising at least two light-induced reaction steps, the fluorophore being formed and the dye acquiring its fluorescent properties only after the last of the reaction steps. Finally, in all methods according to the present disclosure, scanning of the sample or a section of the sample is carried out with an intensity distribution of excitation and/or fluorescence inhibition light comprising an intensity minimum.

The first method according to the present disclosure comprises the introductory steps of:
1. Selecting a fluorescent dye which, initially, is in a protected, non-fluorescent form and which can be converted from the protected to an activated, fluorescent form, i.e., photoactivated, by illumination with activation light. Here, the term ‘fluorescent’ means that the dye can be excited with light of suitable wavelength to emit fluorescent light, whereas this is not possible in the non-fluorescent form. According to the present disclosure, the fluorescent dye is selected such that the photoactivation occurs in at least two respective light-induced reaction steps.
2. Labeling the structure of interest in the sample with the selected dye, for which fluorescent labeling methods known in the prior art can be used, including immunofluorescent labeling techniques.

The further steps of the method may be performed once or repeatedly and include:
3. Photoactivation of a (small) subset of the fluorescent dye from the protected, non-fluorescent form into the activated, fluorescent form by illuminating the sample with activation light. The subset may be spatially defined and may include, e.g., molecules of the fluorescent dye within a section or plane of the sample, but the subset may also be statistically defined and may include a random selection of all molecules. In particular, the subset may comprise individual molecules that are spatially separated from each other.
According to the present disclosure, photoactivation entails an activation reaction comprising at least two light-induced reaction steps, wherein the fluorophore is formed and the dye acquires its fluorescent properties only after the last of the reaction step. Photoactivation greatly increases the fluorescence quantum yield of the dye, i.e., the probability that an electronically excited dye molecule will release its excitation energy in the form of a fluorescence photon, rather than through non-radiative processes. However, since the fluorescence quantum yield of the protected form is usually very small but not exactly zero, the fluorescence contrast obtained with photoactivation is limited in practice. Therefore, as a rule, at least a ratio of 1:10, preferably at least 1:100 and ideally at least 1:1000 is aimed for as a fluorescence contrast.
The design of the photoactivation process in two or more reaction steps can be realized by different reaction schemes. One possible design of the photoactivation process consists in the binding of several photolabile protecting groups to functional groups of the fluorescent dye in such a way that the fluorophore can only be formed after the (parallel or sequential) photolytic cleavage of all protecting groups. Potential attachment sites are, in particular, hydroxy, amino, carbonyl and carboxyl groups contained in the fluorophore. Numerous photolabile protecting groups for these functional groups are known, e.g., o-nitrobenzyl groups, benzoin groups, phenacyl groups, coumaryl and arylmethyl groups, benzoin groups, arylsulfonamides, and substituted variants of these groups. For a comprehensive review, it is referred to the review article “Photoremovable Protecting Groups in Chemistry and Biology: Reaction Mechanisms and Efficacy” by P. Klan et al. in Chem. Rev. 113, 119 (2013). Protecting groups of the type mentioned are also suitable for the preparation of photoactivatable fluorescent dyes, and examples of protected rhodamine and fluorescein dyes are known to the skilled person from the prior art [see, for example, “Specific protein labeling with caged fluorophores for dual-color imaging and super-resolution microscopy in living cells,” S. Hauke et al., Chem. Sci. 8, 559 (2017)].
While most photolabile protecting groups are cleavable with UV light, there is also an increasing number of protecting groups that can be cleaved in the visible spectral range. These include, in particular, coumaryl protecting groups that can be cleaved with blue light (400-500 nm), and boron dipyrromethene (BODIPY)-derived protecting groups (see, for example, “BODIPY-Derived Photoremovable Protecting Groups Unmasked with Green Light,” P. P. Goswami et al, J. Am. Chem. Soc. 137, 3783 (2015), which are cleavable with green light (>500 nm). The wide spectral range of protecting groups now available also allows selective cleavage of different protecting groups from a molecule, see “Wavelength-Selective Cleavage of Photoprotecting Groups: Strategies and Applications in Dynamic Systems,” M. J. Hansen et al., Chem. Soc. Rev. 44, 3358 (2015).
The cleavage of the protective groups can be performed with monochromatic activation light. In particular, if the photolabile protecting groups are chemically identical or at least cleavable with light of the same wavelength, the process can be implemented with only a single activation light source. This activation light source may be a continuous light source or a pulsed light source. In the latter case, particularly when ultrashort pulse lasers are used as the activation light source, photoactivation may also be initiated by two-photon or multiphoton absorption. Alternatively, however, the activation light may include multiple wavelengths, particularly from multiple light sources, if the protecting groups are different and have spectrally different absorption characteristics.
The large variability of the protecting groups allows the design of the photoactivatable fluorescent dye to be optimized case-by-case to induce as little photoactivation by the fluorescence inhibition or stimulation light as possible while, at the same time, providing the largest possible absorption cross-section for the activation light. In this regard, it may be advantageous that the protecting groups differ from each other chemically and in their spectral absorption characteristics. In this case, the activation light can also comprise multiple wavelength components such that protecting groups that are chemically different and absorb in different spectral ranges can be cleaved. The different wavelengths of the activation light can be applied simultaneously or successively.
In the variants mentioned, the cleavage of the several protecting groups can take place simultaneously, i.e., at the same time, or one after the other. A strictly sequential sequence of the two light-induced reaction steps also occurs in another, advantageous variant of the method, in which a so-called tandem reaction is used (which does not necessarily have to result in the cleavage of a protective group). Tandem reactions are reaction sequences consisting of several independent reaction steps (but not mechanistic steps) that proceed in sequence. While, according to the common understanding, tandem reactions include reactions that occur spontaneously one after the other, in the context of this document the term shall also include reactions whose steps are initiated individually and sequentially—in particular by the action of light. According to the present disclosure, the fluorescent form is produced only after the second (or a further) light-induced stage of the tandem reaction. An example for one such tandem reaction was given by A. Paul et al. in “o-Hydroxycinnamate for Sequential Photouncaging of Two Different Functional Groups and its Application in Releasing Cosmeceuticals,” Org. Biomol. Chem. 33 (2019):

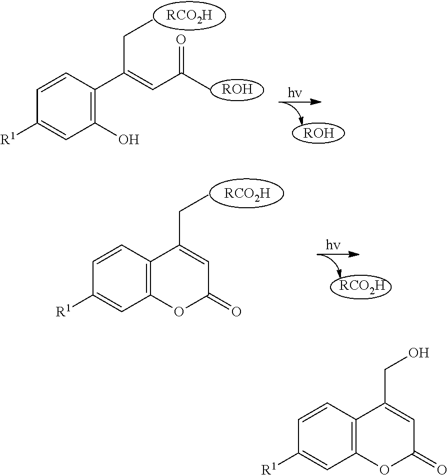

Individual stages of the tandem reaction can also be reversible, so that the initial state or an intermediate state of photoactivation is restored if subsequent reaction stages are not triggered.
4. Forming intensity distributions of excitation light and fluorescence inhibition light in the sample, wherein the intensity distribution of the fluorescence inhibition light comprises a local intensity minimum. As is usual in STED microscopy, the intensity distribution of the excitation light is preferably formed in the form of a diffraction-limited focused light spot whose (central) intensity maximum is spatially superimposed on the intensity minimum of the fluorescence inhibition light. In principle, however, other intensity distributions of the excitation light are also conceivable and suitable for carrying out the method, e.g., a homogeneous distribution of excitation light in the sample.
The term fluorescence inhibition light refers to any type of light that is capable of preventing, reducing, or completely suppressing the fluorescence of the activated fluorescent dye molecules when illuminated with excitation light. In particular, fluorescence inhibition light may be stimulation light known from STED microscopy that induces stimulated emission of electronically excited dye molecules, thereby converting the dye molecules (back) to the electronic ground state and preventing them from spontaneous fluorescence emission. As known from RESOLFT microscopy, fluorescence inhibition light may also induce light-induced chemical reactions, in particular isomerization, cyclization/cycloreversion reactions, which are accompanied by modulation of fluorescence emission.
The superposition of the excitation light with the fluorescence inhibition light results in a narrowing of the range in which fluorescent dye molecules illuminated with excitation light can emit fluorescence light, and thus in an effective PSF that is reduced in size. The resolving power Δ is understood here, according to common practice, as the distance between two identical point objects at which they can still be separated and imaged as separate objects. The resolving power achieved during scanning depends on the intensity of the fluorescence inhibition light and is given—as known to the skilled person from STED and RESOLFT microscopy—by:

$$Δ = \frac{λ}{2 n sin α \sqrt{1 + \frac{I}{{I}_{s} ( λ )}}} \qquad ( 2 )$$

wherein λ is the wavelength of the fluorescence inhibition light, n is the refractive index of an optical material in which the intensity distribution of the fluorescence inhibition light is formed, a is half the aperture angle of an optical arrangement with which the sample is illuminated, I is the intensity of the fluorescence inhibition light or stimulation light at the maximum of the intensity distribution, and Iₛ(λ) is a dye-specific saturation intensity. This saturation intensity is usually defined by the fact that illuminating the dye with fluorescence inhibition light of intensity I=Iₛ reduces the photon number or intensity of the fluorescent light emitted by the dye by a factor of 2.
The forming of the intensity distributions shall not be understood as a completed step, after which the intensity distributions are reverted; rather, the intensity distributions may remain formed during the subsequent scanning step.
5. Scanning the sample or a section of the sample with the previously formed intensity distribution of the fluorescence inhibition light comprising a local intensity minimum at a sequence of scanning positions, wherein an intensity or a photon number of fluorescence light is detected at each scanning position and associated with the scanning position. In this case, the scanning is preferably performed along a regular, in particular a Cartesian or a hexagonal grid; however, this is not mandatory. In particular, scanning can also be performed adaptively, e.g., by scanning the sample or the section of the sample along a grid with a relatively large step size and by scanning with a smaller step size (which may be adapted to the structure) only in those areas in which fluorescence emission is detected. If the areas containing the structure can be identified in advance (e.g., from an epifluorescence image of the specimen or of the section of the specimen), scanning outside these areas can also be skipped completely.
6. Generating a high-resolution raster image of the structure from the associated photon numbers or intensities of the fluorescent light and the scanning positions, wherein a brightness value is assigned to each image pixel of the raster image. In the simplest case, there is a direct one-to-one relationship between the scanning positions on the one hand and the image pixels of the raster image on the other hand, i.e., the photon number detected at the corresponding scanning position or the (digitized) intensity value is directly assigned to each image pixel as a brightness value. However, the numbers of the scanning positions and the image pixels do not necessarily have to match; a combination of the detected photon numbers or intensities from several scanning positions to form one image pixel is also possible as is an interpolation of image pixels from the photon numbers or intensities of neighboring scanning positions.
To increase the dynamic range of the raster image, to highlight weakly fluorescent structures, or to adapt the brightness value to the display characteristics of a display device or the perception characteristics of the human eye, the brightness value may optionally be a (monotonic) function of the photon number or intensity value, e.g., a logarithmic or gamma function.

In a preferred embodiment of the method, the fluorescence inhibition light specifically exhibits a toroidal intensity distribution with a local intensity minimum as a central zero, while the excitation light is typically formed as a diffraction limited, Gaussian focus such that the minimum of the fluorescence inhibition light and the maximum of the excitation light coincide spatially. Methods for the formation of such a toroidal intensity distribution are known to the skilled person from the prior art; the placement of a helical phase plate (vortex phase plate) in the light beam of the fluorescence inhibition light shall be mentioned as an example. In this embodiment, the intensity distributions of the excitation light and fluorescence inhibition light are essentially complementary to each other, i.e., at points of high intensity of the excitation light, the intensity of the fluorescence inhibition light is low and vice versa.

In a parallelized variant of this embodiment, several, possibly even very many, corresponding intensity distributions of excitation and fluorescence inhibition light may alternatively be generated by pairwise interference of four excitation and fluorescence inhibition light beams, forming two, mutually orthogonal standing waves each [see “Nanoscopy with more than 100,000 ‘doughnut’”, A. Chmyrov et al. in Nature Meth. 10, 737 (2013)]. Here, the standing waves of the excitation and fluorescence inhibition light are (phase) shifted with respect to each other, resulting in essentially complementary intensity distributions as well.

For positioning the intensity distributions of excitation and fluorescence inhibition light at the scanning positions, a beam deflection device arranged in the beam path may be used, which can be implemented with galvo mirrors, for example. Alternatively, for higher scanning speeds and, in particular, irregularly arranged scanning positions, electro-optical or acousto-optical deflectors are suitable, which do not require any moving parts and allow particularly fast deflection of the light beams.

The detection of the fluorescent light may be performed with a point detector, a detector array or a camera, depending on the type of intensity distributions of excitation and fluorescence inhibition light. Due to their sensitivity, avalanche photodiodes or avalanche photodiode arrays operated in photon counting mode are particularly suitable, but also (hybrid) photomultipliers or integrating detectors such as CCD and sCMOS cameras. In the case of point illumination with excitation light and detection by means of a point detector, this is preferably arranged behind a confocal pinhole to suppress stray and background light. However, even in the case of point illumination, the fluorescent light may advantageously be registered with a detector array.

Illuminating the sample with the excitation and the fluorescence inhibition light and detecting the fluorescence at the scanning positions may be repeated as needed, e.g., to improve the signal-to-noise ratio or to record time series. If necessary, additional dye molecules may be photoactivated prior to rescanning, e.g., to replace dye that has faded during scanning. Alternatively, it may be desired to activate a completely different subset of the fluorescent dye. In this case, the (remaining) fluorescent dye molecules must first be deactivated again, which, in the simplest case, can be accomplished by bleaching with intense excitation light.

The present disclosure further relates to a method for localizing single molecules of a fluorescent dye in a sample. The method comprises photoactivating individual molecules of a fluorescent dye in an activation reaction comprising two reaction steps, scanning these molecules with an intensity distribution of an excitation light and an intensity distribution of the fluorescence inhibition light, and detecting the fluorescence at each scanning position. In this scanning, one of the two intensity distributions may remain stationary, at least one of the two intensity distributions is sequentially positioned at a plurality of scanning positions. From the fluorescence intensities and the scanning positions, new position estimates of the fluorescence molecules are calculated as updates to the previous position estimates. When stationary fluorescent dye molecules are localized the new position estimates are improved position estimates that estimate the actual position of the fluorescent dye molecule of interest with less uncertainty. If moving fluorescent dye molecules are observed, the values may represent a new, changed position of the respective fluorescent dye molecule. The steps of the method are in detail as follows:
1. Selecting a fluorescent dye which is such that it is initially present in a protected, non-fluorescent form and which can be converted from the protected to an activated, fluorescent form, i.e., photoactivated, by illumination with activation light. According to the present disclosure, the fluorescent dye is selected such that the photoactivation comprises at least two respective light-induced reaction steps.

The method further comprises a first group of process steps:
2. Photoactivation of one or more molecules of the fluorescent dye which are spaced apart from each other by a minimum distance d, from the protected, non-fluorescent form into the activated, fluorescent form by illuminating the sample with activation light. According to the present disclosure, as in the first-mentioned method according to the present disclosure, photoactivation is carried out by an activation reaction comprising at least two light induced reaction steps, wherein the fluorophore is formed and the dye acquires its fluorescent properties only after the last of the reaction steps. With regard to the requirements for and the design of the photoactivation process, the statements in the description of the first process according to the present disclosure also apply here.
To ensure the minimum distance d between several activated dye molecules, the spatial density of activated dye molecules must be precisely controlled during illumination with activation light. For this purpose, the illumination parameters of the activation light, i.e., in particular, the illumination duration and intensity, can be determined once by trial and error to ensure the requirement of the presence of spatially isolated dye molecules for a given sample and under given imaging conditions. However, if the sample can be observed continuously during photoactivation, e.g., by exposing it to excitation light while detecting fluorescent light photoactivation may also be performed under direct control; the exposure with activation light can be stopped when the density of molecules reaches a threshold. If the activation takes place in a locally confined area, the activation can be stopped when the observed fluorescence signal exceeds a threshold value, which can also be 0 (zero). Controlled activation is particularly advantageous when, during the course of data acquisition, the total amount of dye decreases due to photobleaching and the illumination duration or intensity must be increased to maintain a constant density of activated dye molecules, or to activate a dye molecule in a localized area. The distance between activated dye molecules can alternatively or additionally be controlled by illuminating the sample with an illumination pattern, for example in the form of illumination dots on a grid, rather than over a large area and homogeneously with activation light.
3. Determining initial position estimates of one or more activated dye molecules as starting values for the subsequent scanning and detection step of the method. The initial position estimates are determined in a manner such that they have an uncertainty of at most d/2 so that they can be unambiguously assigned to one of the activated dye molecules that are spaced apart by at least the distance d from each other.
The initial position estimates can basically be determined in different ways. In particular, the position estimates may be determined from a fluorescence image of the sample or a section of the sample by localizing the activated dye molecules. A fluorescence image taken with a camera or by confocal laser scanning is particularly suitable for this purpose. In a more specific variant of the method, the initial position estimates may also be determined by means of STED microscopy, which may allow a higher spatial density of activated dye molecules.
The determination of initial position estimates by means of confocal or STED microscopy may also be varied in such a way that entire image fields are not scanned in order to (subsequently) identify and localize individual dye molecules in these images, but that the sample is scanned until a single, activated dye molecule is detected and its position can be approximately determined from the respective scanning position. The subsequent scanning and detection step may then immediately follow the detection of a dye molecule in each case, so that these process steps do not necessarily have to be carried out strictly separately from one another but can also be carried out alternately. Likewise, photoactivation may also be coupled with scanning. For this purpose, the sample may be scanned point by point and line by line with activation and excitation light (simultaneously or as a fast sequence) until a single activated dye molecule is detected. The scanning and detection step may again immediately follow the detection of a dye molecule.
In particular, if the activation is performed in a spatially confined area by means of a diffraction-limited focused activation light beam, the determination of the initial position estimate may also be performed directly from the position of the known focus point of the activation light beam; this position is then used as the initial position estimate.
4. Forming an intensity distribution of an excitation light and an intensity distribution of a fluorescence inhibition light in the sample. According to its wavelength, the excitation light is suitable for exciting the activated fluorescent dye to cause fluorescence emission, while the term fluorescence inhibition light, again, comprises any type of light suitable for preventing, reducing or completely suppressing the fluorescence of activated fluorescent dye molecules when illuminated with excitation light. Specifically, the fluorescence inhibition light may be stimulation light that induces stimulated emission of electronically excited dye molecules, thereby converting the dye molecules (back) to the electronic ground state and preventing them from spontaneously emitting fluorescence. Usually, both intensity distributions—or at least the intensity distribution of the excitation light—remain formed during the subsequent scanning step.
According to the present disclosure, at least the fluorescence inhibition light comprises an intensity distribution with a local intensity minimum and prevents or suppresses the emission of fluorescence in areas outside this local intensity minimum. The aim is not primarily to reduce the volume of an effective PSF in a sense to increase the resolution as in STED microscopy, but rather to suppress unwanted fluorescence of dye molecules from focal edge regions or other planes of the sample. Thus, detection of fluorescence can be limited to a fluorescent dye molecule located near the local intensity minimum, and unwanted contributions from other fluorescent dye molecules, particularly from focal edge regions or other planes of the sample, can be suppressed. Thus, with respect to a particular embodiment of the subsequent scanning and localization steps and unlike in STED microscopy, it may even be explicitly desirable to make the intensity minimum as broad as possible and the intensity increase in the vicinity of the intensity minimum as flat as possible, so as to have little effect on the fluorescence emission of a fluorescent dye molecule located in the vicinity of the intensity minimum when the intensity minimum is shifted. Such a broad-zero intensity distribution can be generated, e.g., by phase modulating the fluorescence inhibition light beam with a higher-order vortex phase plate whose phase delay does not vary from 0 to 2π varies with the orbital angle (as is common in STED microscopy), but from 0 to 4π or a higher multiple of 2π. Notwithstanding the foregoing, however, an increase in resolution by the fluorescence inhibition light in the sense of STED microscopy may also be advantageous and desirable in certain embodiments of the method.
Depending on the particular embodiment of the method, the intensity distribution of the excitation light may be designed in different ways, but in particular the intensity distribution of the excitation light may also have a local intensity minimum. Preferred embodiments of the method include the combination of point-focused excitation light with a toroidal intensity distribution of fluorescence inhibition light and the combination of two toroidal intensity distributions of excitation and fluorescence inhibition light.

Finally, the process includes a second group of process steps:
5. Scanning the sample or a section of the sample with one of the previously formed intensity distributions comprising an intensity minimum at a sequence of scanning positions, wherein a photon number or an intensity of fluorescent light is detected at each scanning position and associated with the scanning positions. These value pairs formed from the photon numbers or fluorescence intensities and the scanning positions form the data basis for the subsequent localization step. The scanning positions are chosen in such a way that there are at least two scanning positions at a distance of less than d/2 around the estimated position value of each dye molecule to be localized. The scanning is performed with an intensity distribution that comprises a local intensity minimum—ideally an intensity zero. This may be an intensity distribution of excitation light or of fluorescence inhibition light. In either case, the sample or section of the sample is exposed to excitation light at each scanning step. If the scanning is done with fluorescence inhibition light, the intensity distribution of the excitation light may be stationary. If the fluorescence inhibition light is stimulation light, the sample or the section of the sample is exposed to fluorescence inhibition light, i.e., the stimulation light, in each scanning step. If the fluorescence inhibition light is a switching light that switches fluorophores to a (meta-)stable, non-fluorescent state, it may be sufficient to apply fluorescence inhibition light to the sample once at the beginning of the scanning step. In this respect, the first step of the second group of method steps is not strictly separate from the last step of the first group.
In particular, when high intensities of the light forming the intensity minimum are applied, a steep intensity gradient is formed starting from the intensity minimum, so that the fluorescence emission of a dye molecule located near the intensity minimum changes significantly even with a small shift of the scanning position. In this process, the fluorescence emission may increase (if scanning is performed with excitation light) or decrease (if scanning is performed with fluorescence inhibition light) with increasing distance of the dye molecule from the intensity minimum. The strong dependence of the fluorescence emission on the scanning position, regardless of its direction, forms the basis for improving the position estimate in the localization step.
In a preferred embodiment, both the excitation light and the fluorescence inhibition light comprise intensity distributions with spatially superimposed intensity minima, for example as toroidal (donut-shaped) intensity distributions. In this embodiment, scanning is further preferably performed with the intensity distribution of the excitation light, while the position of the intensity distribution of the fluorescence inhibition light is stationary during scanning at the scanning positions, each of which is associated with an activated fluorescent dye molecule, and thus has no influence on the change of the fluorescence signal at the different scanning positions. Nevertheless, the intensity distribution of the fluorescence inhibition light may also be co-located during scanning as it passes from one dye molecule to another. Intensity distributions that exhibit only a small intensity variation within the scanning range of a dye molecule and that may be generated, e.g., with higher-order vortex phase plates (see above) are particularly suitable for the fluorescence inhibition light. In this embodiment, the purpose of the fluorescence inhibition light is solely to suppress contributions to the detected fluorescence from dye molecules outside the intensity minima, thus enabling the localization of the scanned molecules or making it more precise by eliminating interfering contributions. In principle, scanning with both intensity distributions is also possible, but then it must be taken into account that the fluorescence of the dye molecules is influenced by both intensity distributions during scanning. Advantages may arise in particular if fluorescence excitation, suppression with stimulation light and detection are performed using temporal gates, especially if fluorescence detection is limited to a very short time window, e.g., less than half, preferably less than a quarter, more preferably less than a tenth of the fluorescence lifetime after the time at which fluorescent light emitted from the observation volume at the temporal maximum of the excitation pulse would generate a signal on the detector.
In another preferred embodiment, the combination of a Gaussian-focused spot of excitation light with a toroidal (donut-shaped) intensity distribution of fluorescence inhibition light as known from STED microscopy is used. Here, only the fluorescence inhibition light comprises an intensity distribution with an intensity minimum, and scanning is performed accordingly with the fluorescence inhibition light, while the position of the intensity distribution of the excitation light during scanning at the scanning positions, each associated with an activated fluorescent dye molecule, is preferably stationary and thus has no influence on the change of the fluorescence signal at the various scanning positions. The intensity distribution of the excitation light is repositioned only during the transition from one dye molecule to another or when tracking the movement of a dye molecule in the sample.
The minimum number of two scanning positions per dye molecule to be localized allows highly accurate localization of the dye molecule in one dimension. If, on the other hand, localization in two dimensions is desired, the minimum number of scanning positions increases to three per dye molecule. It is advantageous to arrange the scanning positions so that the dye molecule is located within the triangle formed by the three scanning positions; however, this is not strictly required. For localization in three dimensions, the minimum number increases to four scanning positions per dye molecule. Again, it is advantageous (but not mandatory) to arrange the scanning positions such that the dye molecule is located within a tetrahedron defined by the four scanning positions. In practice, increasing the number of scanning positions beyond these minimum numbers may be useful to further improve localization accuracy. In particular, it may be advantageous to use as one scanning position the position estimate determined prior to the respective execution of the second group. This ensures that a position estimate is unambiguously obtained from the registered fluorescence signals, i.e., that the measured fluorescence signals cannot be ambiguous.
6. In the final localization step, improved position estimates of the activated dye molecules are determined from the associated photon numbers or intensities and scanning positions. In the simplest case, this is done by summing the position vectors of the scanning positions weighted by the photon numbers or fluorescence intensities. Other common, much more accurate methods use a maximum likelihood estimator (MLE), e.g., and are known from the state of the art for MINFLUX nanoscopy.

According to the present disclosure, the value of d must be selected in such a way that initial position estimates can be unambiguously associated with the activated fluorescent dye molecules and that the detected fluorescent light at each scanning position originates from only one activated molecule of the fluorescent dye at a time. On the one hand, this means that the minimum distance between the activated dye molecules must not fall below the optical resolving power of the method used to determine the initial position estimates, such that due to the optical diffraction limit, d≥250 nm is given. Only in the case that the initial position estimates are determined with an already higher resolution method (e.g., by STED microscopy), the value of d may also be selected smaller. However, it must also be taken into account that the minimum distance between the activated dye molecules is also limited to small values by the fact that only one activated molecule of the fluorescent dye may contribute to the detected fluorescence signal at a time when scanning the sample or the section of the sample with the light distribution having an intensity minimum. Therefore, only in special variants of the method—e.g., when scanning with a combination of a Gaussian-focused light spot of excitation light and a toroidal (donut-shaped) intensity distribution of fluorescence inhibition light—is it possible to reduce the value from d to below 250 nm.

When implementing the method according to the present disclosure, the sequence of scanning positions does not necessarily have to be fully specified before the start of scanning but can also be successively amended. In a particularly advantageous manner, the sequence of scanning positions associated with a dye molecule may be amended while taking into account the preceding determination step or steps. Since the uncertainty in the position estimate is substantially reduced by a localization step, scanning positions may subsequently be arranged much more densely around the dye molecule to be localized. Scanning at these newly established scanning positions, together with a (further) increase in the intensity of the light used for scanning, leads to improved position estimates in the next determination step. Repeated application of these steps allows localization of the dye molecule down to a few nanometers.

In a further embodiment of the method according to the present disclosure, the determination of position estimate values of a single activated dye molecule is carried out repeatedly, for example at fixed intervals. If the method is applied to track the movement of a dye molecule in a sample, the scanning positions for a determination step are determined on the basis of the position estimate value of the preceding determination step, as may also be the case in the localization of non-moving dye molecules. In the case of tracking molecules, the spacing of the scanning positions is advantageously adapted to the speed and type of movement of the dye molecules, in particular to the extent they are known from the preceding determination steps. If the movement is fast and random, large distances between the scanning positions are selected so that the molecule is reliably located in each case within the range defined by the scanning positions in which the position of a molecule can be estimated. If, on the other hand, the motion is slow and directional, the spacing of the scanning positions may be chosen smaller but subject to the condition that the molecule is reliably within the range defined by the scanning positions in which the position of a molecule can be estimated. In both cases, the center of the set of scanning positions is moved to the location where the molecule is expected to be during the next sequence of scanning steps. In the case of random motion, this is the location corresponding to the most recently determined position estimate. From the successively determined position estimate values, a trajectory of the dye molecule may be reconstructed, visualized and, if necessary, further analyzed. When using the dye as a marker for a biomolecule, e.g., a protein or a lipid, such trajectories are suitable for studying dynamic cellular processes in which the labeled biomolecule is involved. In addition to the high spatial resolution, the method according to the present disclosure also allows a considerably faster determination of the positions of single molecules than is possible with methods known from the prior art, thus extending the applicability of single molecule tracking to fast dynamic processes.

The entire process of tracking individual dye molecules may be repeated for additional dye molecules. Such repetition may begin with activation. If, after tracking one molecule, others are in an activated state, it may be sufficient to repeat only the second set of process steps, now on another dye molecule. Also, from the set of trajectories, a high-resolution image of a structure in the sample may eventually be reconstructed. For example, values of diffusion constants can be determined at local resolution. The spatial distribution of these values may then map a structure.

In another embodiment of the method according to the present disclosure, the localizations are used to reconstruct a high-resolution image of a structure in the sample labeled with the fluorescent dye, e.g., in the form of a two-dimensional histogram. This type of image reconstruction is known from STORM and PALM microscopy. However, in order to generate a high-resolution image of the structure that is as spatially continuous as possible, it is necessary to determine the positions of a sufficiently large number of fluorescent dye molecules—typically several thousand. For this purpose, the method steps from photoactivation to localization of the activated dye molecules are applied several times in order to localize the desired high number of molecules of the fluorescent dye. In this process, the respective active dye molecules must be converted to a non-fluorescent state between the repetitions. In the simplest case, this may be achieved by irreversibly bleaching the active molecules with intense excitation light. Provided the photoactivation is reversible, the activated molecules may also be restored to the non-fluorescent state with light of suitable wavelength.

Finally, the present disclosure further comprises a method for localizing single molecules of a fluorescent dye in a sample, the method combining features of the first two methods of the present disclosure. The steps of the method are in detail as follows:
1. Selecting a fluorescent dye which is such that it is initially present in a protected, non-fluorescent form and which can be converted from the protected to an activated, fluorescent form, i.e., photoactivated, by illumination with activation light. According to the present disclosure, the fluorescent dye is selected such that the photoactivation comprises at least two respective light-induced reaction steps.

The method further comprises the group of method steps:
2. Photoactivation of one or more molecules of the fluorescent dye which are spaced apart by a minimum distance d, from the protected, non-fluorescent form into the activated, fluorescent form by illuminating the sample with activation light, wherein the photoactivation is carried out by an activation reaction comprising at least two light-induced reaction steps.
3. Forming an intensity distribution of an excitation light and an intensity distribution of a fluorescence inhibition light in the sample, wherein the intensity distribution of the fluorescence inhibition light comprises a local intensity minimum. Usually, both intensity distributions—but at least the intensity distribution of the excitation light—remain formed during the subsequent scanning step.
4. Scanning the sample or a section of the sample with the intensity distribution of the fluorescence inhibition light comprising a local intensity minimum at a sequence of scanning positions which are spaced from one another by a distance of not more than d/2, wherein at each scanning position an intensity or a photon number of fluorescence light is detected and associated with the scanning position. Therein, the scanning is preferably performed along a regular grid, in particular a Cartesian grid or a hexagonal grid; however, this is not mandatory. In particular, scanning may also be performed adaptively, e.g., by scanning the sample or the section of the sample along a grid with a relatively large step size and only in those areas in which fluorescence emission is detected, scanning is performed with a smaller step size, possibly adapted to the structure. If the areas containing the structure can be identified in advance (e.g., from an epifluorescence image of the specimen or of the section of the specimen), scanning outside these areas can also be dispensed with completely.
5. Localizing activated dye molecules from the associated photon numbers or intensities of the fluorescent light and the scanning positions with an uncertainty of at most d/10 in at least one spatial direction,

According to the present disclosure, the value of d should be chosen such that initial position estimates can be unambiguously associated with the activated fluorescent dye molecules and that the detected fluorescent light at each scanning position originates from only one activated molecule of the fluorescent dye at a time.

Moreover, a first aspect of the present disclosure relates to a method for localizing single molecules of a fluorescent dye in a sample comprising the method step of selecting a fluorescent dye that is convertible from a protected, non-fluorescent form to an activated, fluorescent form by illumination with an activation light. The method comprises a first group of method steps comprising the steps of photoactivation of one or more molecules of the fluorescent dye, which are spaced apart by a minimum distance d from each other, from the protected, non-fluorescent form into the activated form by illumination with activation light, determining initial position estimates of one or more activated dye molecules with an uncertainty of no more than d/2, and forming an intensity distribution of an excitation light and an intensity distribution of a fluorescence inhibition light in the sample, wherein at least the intensity distribution of the fluorescence inhibition light comprises a local intensity minimum, wherein also the intensity distribution of the excitation light may comprise an intensity minimum; and a second group of method steps comprising the steps of scanning the sample or a section of the sample with one of the intensity distributions comprising an intensity minimum at a sequence of scanning positions, the sequence containing subsets each comprising at least two scanning positions which are arranged at a distance of less than d/2 around the position estimate of an activated dye molecule associated with the subset, detecting a photon number or an intensity of fluorescent light at each scanning position of the sequence, and associating the photon number or the intensity with the respective scanning position, and determining a new position estimate for each of the activated dye molecules associated with a subset from the associated photon counts or intensities of fluorescent light and the scanning positions, wherein the value of d is such that the initial position estimates can be unambiguously associated with the activated fluorescent dye molecules and that the detected fluorescent light at each scanning position originates from only a single activated molecule of the fluorescent dye in each case, the fluorescent dye is selected such that the photoactivation comprises at least two respective light-induced reaction steps.

According to an embodiment of the first aspect, d≥250 nm.

According to a further embodiment of the first aspect, the intensity distribution of the excitation light comprises a local intensity minimum and that the sample or the section of the sample is scanned with the intensity distribution of the excitation light.

According to a further embodiment of the first aspect, the intensity distribution of the excitation light comprises a local intensity maximum, wherein the intensity distributions of the fluorescence inhibition light and the excitation light are substantially complementary to each other.

According to a further embodiment of the first aspect, the scanning is performed with both intensity distributions.

According to a further embodiment of the first aspect, the scanning positions of the sequence of scanning positions are arranged on circular paths, spiral paths or spherical shells.

According to a further embodiment of the first aspect, the second group of method steps is carried out repeatedly.

According to a further embodiment of the first aspect, an overall intensity of the intensity distribution of the excitation light and/or the fluorescence inhibition light comprising a local intensity minimum is increased between the repetitions and the scanning positions of the subsets are shifted in the direction of the respective current position estimate of the associated activated fluorescence molecule.

According to a further embodiment of the first aspect, the last determined position estimate has an uncertainty of at most d/10 and preferably of at most d/30 in at least one spatial direction.

According to a further embodiment of the first aspect, a movement of individual molecules of the fluorescent dye in a sample is tracked.

According to a further embodiment of the first aspect, the overall intensity of the intensity distribution of the excitation light and/or the fluorescence inhibition light comprising a local intensity minimum is reduced between two repetitions and the scanning positions of the subsets are shifted in the direction of a position estimate of the associated activated fluorescence molecule which is determined by temporal extrapolation. By reducing the overall intensity of the intensity distribution, the catch area, that is, the area in which the position the tracked fluorescence dye can be unambiguously deduced by an estimator from the scanning positions and the associated photon counts or fluorescence intensities, is enlarged. Thereby, losing fluorescence dyes during tracking is avoided.

According to a further embodiment of the first aspect, the overall intensity of the intensity distribution of the excitation light and/or the fluorescence inhibition light comprising a local intensity minimum is increased between two repetitions and the scanning positions of the subsets are shifted in the direction of a position estimate of the associated activated fluorescence molecule which is determined by temporal extrapolation.

According to a further embodiment of the first aspect, the first and the second group of method steps are carried out repeatedly as a whole, wherein between the repetitions the respective activated dye molecules are converted into a non-fluorescent state.

According to a further embodiment of the first aspect, a spatially high-resolution image of a structure in the sample is reconstructed from the localizations of the individual molecules of the fluorescent dye.

A second aspect of the present disclosure relates to a method for localizing single molecules of a fluorescent dye in a sample, comprising the method step of selecting a fluorescent dye that is convertible from a protected, non-fluorescent form to an activated, fluorescent form by illumination with an activation light, and a group of method steps comprising the steps of photoactivation of one or more molecules of the fluorescent dye, which are spaced apart by a minimum distance d from each other, from the protected, non-fluorescent form into the activated form by illumination with activation light, forming an intensity distribution of an excitation light and an intensity distribution of a fluorescence inhibition light in the sample, wherein the intensity distribution of the fluorescence inhibition light comprises a local intensity minimum, scanning the sample or a section of the sample with the intensity distribution of the fluorescence inhibition light comprising an intensity minimum at a sequence of scanning positions which are spaced apart from one another by a distance of not more than d/2; detecting a photon number or an intensity of fluorescent light at each scanning position of the sequence, and associating the photon number or the intensity with the respective scanning position, and localizing activated dye molecules from the associated photon numbers or intensities of the fluorescent light and the scanning positions with an uncertainty of at most d/10 in at least one spatial direction, wherein the value of d is such that the detected fluorescent light at each scanning position originates from only a single activated molecule of the fluorescent dye, wherein the fluorescent dye is selected such that the photoactivation comprises at least two respective light-induced reaction steps.

According to an embodiment of the second aspect, d≥250 nm.

According to a further embodiment of the second aspect, the intensity distribution of the excitation light comprises a local intensity maximum, wherein the intensity distributions of the fluorescence inhibition light and the excitation light are substantially complementary to each other.

According to a further embodiment of the second aspect, the scanning positions are arranged on a regular grid.

According to a further embodiment of the second aspect, the group of method steps is carried out repeatedly, wherein between the repetitions the respective activated dye molecules are converted into a non-fluorescent state.

According to a further embodiment of the second aspect, a spatially high-resolution image of a structure in the sample is reconstructed from the locations of the activated dye molecules determined by the localization.

A third aspect of the present disclosure relates to a method for generating spatially high-resolution images of a structure in a sample comprising the method steps of selecting a fluorescent dye that is convertible from a protected, non-fluorescent form to an activated, fluorescent form by illumination with an activation light, labeling the structure with the fluorescent dye, as well as the following method steps carried out once or repeatedly: photoactivation of a subset of the fluorescent dye from the protected, non-fluorescent form into the activated form by illumination with activation light, forming an intensity distribution of an excitation light and an intensity distribution of a fluorescence inhibition light in the sample, wherein the intensity distribution of the fluorescence inhibition light comprises a local intensity minimum, scanning the sample or a section of the sample with the intensity distribution of the fluorescence inhibition light comprising an intensity minimum at a sequence of scanning positions, detecting a photon number or an intensity of fluorescent light at each scanning position and associating the photon number or the intensity to the respective scanning position, and generating a high-resolution raster image of the structure from the associated photon numbers or intensities of the fluorescent light and the scanning positions by associating with each image pixel of the raster image a brightness value that is a monotonic function of the photon number or intensity of the fluorescent light detected at the respective scanning position or a respective set of scanning positions, wherein the fluorescent dye is selected such that the photoactivation comprises at least two respective light-induced reaction steps.

According to an embodiment of the third aspect, an intensity distribution, which comprises a local intensity maximum and is substantially complementary to the intensity distribution of the fluorescence inhibition light, is formed by the excitation light and that the scanning is performed together with the excitation light and the fluorescence inhibition light.

According to a further embodiment of the third aspect, the scanning positions are arranged on a regular grid.

According to an embodiment of the first, second or third aspect, the activation light is used to form a plurality of illumination points in the sample.

According to a further embodiment of the first, second or third aspect, the illumination points are arranged on a regular grid.

According to a further embodiment of the first, second or third aspect, a light-induced reaction step is induced by multiphoton absorption.

According to a further embodiment of the first, second or third aspect, all light-induced reaction steps are induced with activation light of identical wavelength.

According to a further embodiment of the first, second or third aspect, one of the light-induced reaction steps is induced with activation light of a different wavelength than another light-induced reaction step.

According to a further embodiment of the first, second or third aspect, at least one of the light-induced reaction steps is a photolytic cleavage of a photolabile protecting group.

According to a further embodiment of the first, second or third aspect, the photolabile protecting group is selected from the group (each unsubstituted or substituted): nitrobenzyl, nitrophenethyl, nitroindolinyl, dinitroindolinyl, nitroveratryl, arylcarbonylmethyl, alkylphenacyl, hydroxyphenacyl, benzoin, hydroxycinnamate, o-nitro-2-phenethyloxy carbonyl, nitroanilide, coumarinyl, aminocoumarinyl, methoxycoumarylmethyl, anthraquinone-2-ylmethoxycarbonyl, (2-naphthyl)methyl, (anthracene-9-yl)methyl, (pyren-1-yl)methyl, (perylen-3-yl)methyl, (phenanthren-9-yl)methyl, o-hydroxyarylmethyl, azide, borondipyrro methenyl.

According to a further embodiment of the first, second or third aspect, the light-induced reaction steps are photolytic cleavage reactions of identical photolabile protecting groups.

According to a further embodiment of the first, second or third aspect, the light-induced reaction steps are photolytic cleavage reactions of different photolabile protecting groups.

According to a further embodiment of the first, second or third aspect, at least two of the light-induced reaction steps are steps of a tandem reaction.

According to a further embodiment of the first, second or third aspect, one step of the tandem reaction is reversible.

A fourth aspect of the present disclosure relates to a use of a fluorescent dye in a method according to the first, second or third aspect, wherein the fluorescent dye is convertible or converted from a protected, non-fluorescent form to an activated, fluorescent form by illumination with an activation light, and wherein the conversion of the dye to the fluorescent form comprises at least two respectively light-induced reaction steps. Further advantageous embodiments of the present disclosure are apparent from the claims, the description and the drawings. The advantages of features and of combinations of several features of the present disclosure described in the description are merely exemplary and may have an alternative or cumulative effect without the advantages necessarily having to be achieved by embodiments according to the present disclosure. Without this altering the subject matter of the appended claims, the following applies with respect to the disclosure content of the original application documents and the patent: further features can be found in the drawings. The combination of features of different embodiments of the present disclosure or of features of different claims is also possible in deviation from the selected back relationships of the patent claims and is hereby suggested. This also applies to such features which are shown in separate figures or are mentioned in the description thereof. These features can also be combined with features of different claims.

Likewise, features listed in the claims may be omitted to form further embodiments of the present disclosure.

The indefinite article “a” used in the patent claims and the description for a feature is to be understood in such a way that, with respect to the number, it can be exactly one or also several implementations of this feature without requiring an explicit use of the adverb “at least”. The features listed in the claims can, if necessary, be supplemented by further features or can also be the only features which the respective method comprises.

### BRIEF DESCRIPTION OF THE FIGURES

FIG. 1 shows a part of a method according to the present disclosure in the form of a flow chart.

FIG. 2 shows photoactivation and scanning steps in a method according to the present disclosure.

FIG. 3 shows a fluorescent dye for use in the methods of the present disclosure.

FIG. 4 illustrates an embodiment of the method comprising scanning positions along a grid and direct image generation.

FIG. 5 illustrates an embodiment of the method comprising the localization of individual fluorescent dye molecules to generate an image.

FIG. 6 shows a tabular overview of various embodiments of the methods according to the present disclosure.

### DESCRIPTION OF THE FIGURES

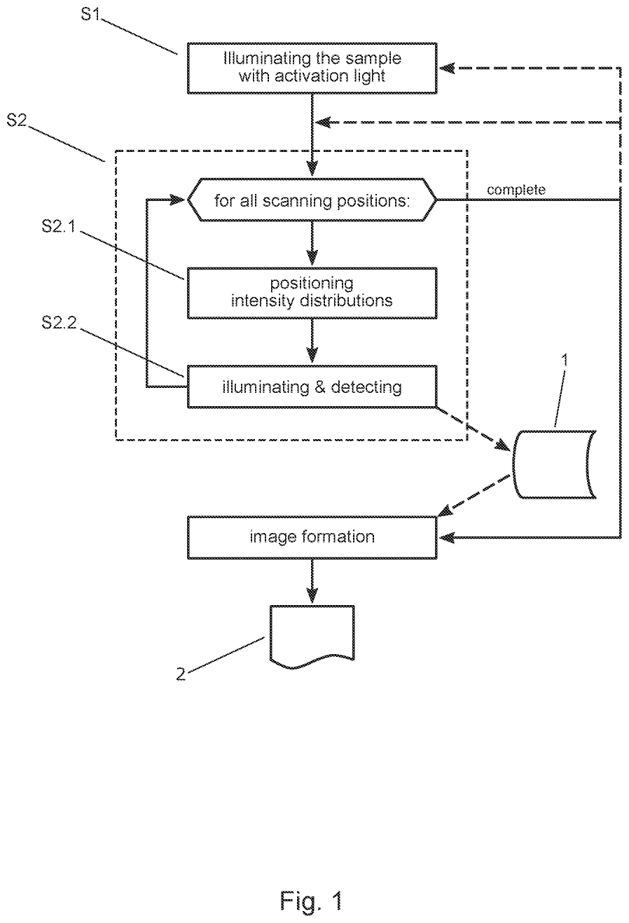

FIG. 1 shows a part of a method according to the present disclosure in the form of a flow chart. First, in a photoactivation step S1, a portion of a fluorescent dye with which a structure in a sample is stained is converted from a protected, initially non-fluorescent form into an activated, fluorescent form by illumination with activation light. According to the present disclosure, this photoactivation is accomplished by at least two light-induced reaction steps. In the subsequent scanning and detection step S2, excitation light that excites the fluorescent dye to emit fluorescent light and fluorescence inhibition light that prevents, reduces or completely suppresses fluorescence emission by the fluorescent dye are positioned at a sequence of scanning positions in the sample (positioning sub step S2.1). At each of the scanning positions where the sample is illuminated, a photon number or an intensity of fluorescent light is detected (scanning and detection sub step S2.2). The detected photon numbers or intensities of fluorescent light are stored in a data memory 1 together with the respective scanning position for later processing.

Optionally, the scanning may be repeated at all or at selected scanning positions. Also optionally, all process steps, i.e., the photoactivation step S1 and the scanning and detection step S2 may be repeated to activate and scan another or a different part of the fluorescent dye with which the sample is labeled.

Finally, in the embodiment shown in FIG. 1, a high-resolution image 2 is generated from the photon numbers or intensities of fluorescent light detected at the scanning positions. The image 2 is, e.g., a raster image whose image pixels reproduce the fluorescence signals detected at each scanning position and thus provide a spatial representation of the structure marked with the dye. Provided that the photoactivation is controlled in such a way that individual, spatially separated molecules of the fluorescent dye are activated, the photon numbers or intensities of the fluorescent light now to be associated with individual molecules may alternatively be used in an intermediate step (not shown) to localize these molecules of the fluorescent dye and the image 2 may be reconstructed from the positions of the localized dye molecules.

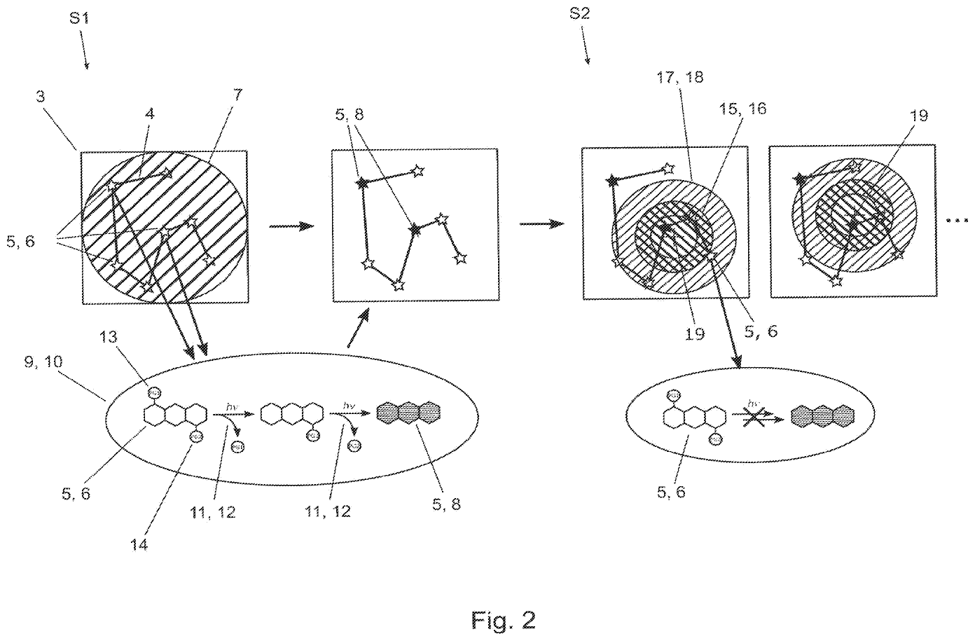

FIG. 2 shows a section 3 of a sample containing a structure 4. The structure 4 is labeled with a fluorescent dye 5, which is initially in a protected, non-fluorescent form 6. In the introductory photoactivation step S1, the section 3 of the sample is illuminated with activation light 7 that converts a small portion of the fluorescent dye 5 into an activated, fluorescent form 8. Photoactivation 9 occurs in a two-step reaction 10, in this case by reaction steps 11 in the form of cleavage 12 of two photolabile protecting groups 13 and 14, forming the activated fluorescent dye 5, 8. After photoactivation 9, the section 3 of the sample is scanned with excitation light 16, here in the form of an intensity distribution 15 exhibiting a local maximum, and with an intensity distribution 17 of fluorescence inhibition light 18 exhibiting a local minimum, at a sequence of scanning positions 19, of which only the first two scanning positions are shown here by way of example. Due to the design of the photoactivation 9 according to the present disclosure in the form of a two-step reaction 10, the fluorescent dye 5 in its protected form 6 is inert with respect to the excitation light 16 and the fluorescence inhibition light 18, so that fluorescent dye 5 illuminated in the protected, non-fluorescent form 6 is not activated during the scanning and detection step S2 with excitation light 16 and fluorescence inhibition light 18.

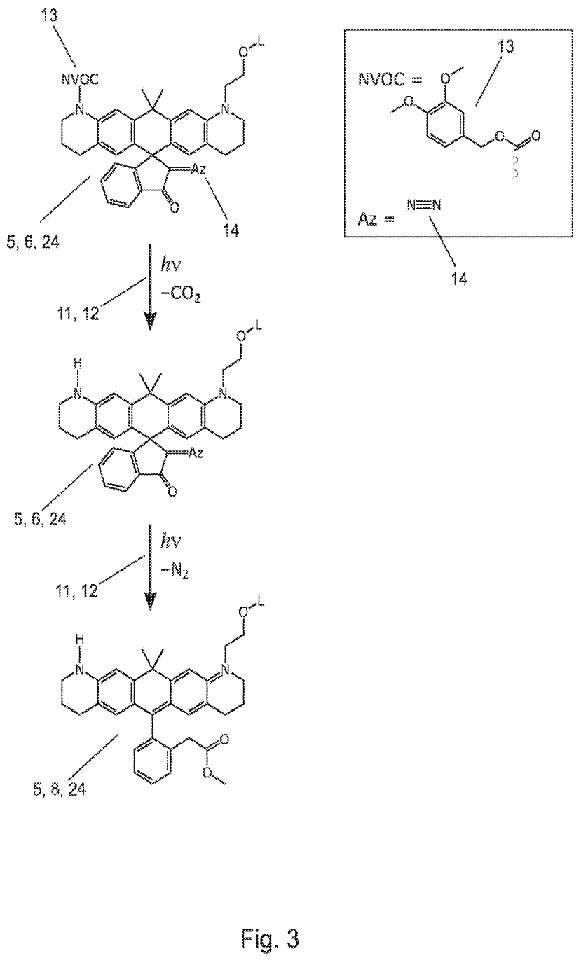

FIG. 3 shows a fluorescent dye 5 derived from caged Q-rhodamine known from the prior art, with a so-called carbopyronine backbone and two photolabile protecting groups 13 and 14 for use in the methods according to the present disclosure. Via the linker group L, the fluorescent dye may be bound to a structure in the sample. The protecting groups 13, 14 prevent the formation of a fluorescent carbopyronine fluorophore at two different positions in the molecule 24: while the o-nitroveratryloxycarbonyl group (NVOC) 13 blocks one of the amino groups, the azide group (Az) 14 fixes the (non-fluorescent) spiro form of the molecule 24. Consequently, the fluorescent dye 5 is present in a protected, non-fluorescent form 6 until both protecting groups 13, 14 are cleaved off. The protecting groups can be cleaved off with UV light with wavelengths in the range of 320 nm to 360 nm, wherein in a reaction step 11 the NVOC group 14 decomposes under decarboxylation (cleavage 12 of CO₂), while the azide protecting group 14 is removed in a further reaction step 11 under cleavage 12 of a nitrogen molecule N₂ and a downstream Wolff rearrangement, thereby forming the activated fluorescent dye 5, 8.

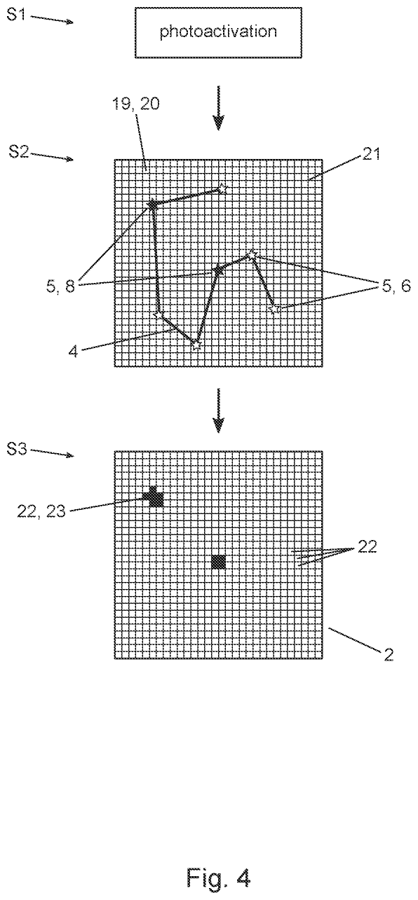

FIG. 4 shows a preferred embodiment of one of the methods according to the present disclosure, in which the scanning positions 19 are arranged on the grid points 20 of a regular, here Cartesian grid 21. As is usual in laser scanning microscopy, the scanning of the sample with excitation and fluorescence inhibition light is performed line by line in the scanning and detection step S2, and the photon number or intensity of the fluorescence light detected at each scanning position 19 is assigned as a brightness value 23 to an image pixel 22 in image 2 corresponding to the respective scanning position 19. In this embodiment, the scanning and detection step S2 and the image generation step S3 do not necessarily occur separately in time; rather, it is advantageous to generate the image 2 while the sample is still being scanned and to display it on a display device.

Non-activated fluorescent dye 5, 6 behaves inertly with respect to the excitation and fluorescence inhibition light and thus is not subject to photoactivation or bleaching during scanning of the sample. By repeatedly performing the photoactivation step S1, the scanning and detection step S2, and the image generation step S3, the method of the present disclosure enables multiple image acquisition of the structure 4 even if the photoactivated fluorescent dye 5, 8 fades during scanning with excitation and fluorescence inhibition light, and thus repeated image acquisition would not be possible with conventional image acquisition methods. The images obtained during the repetitions may be stored individually, e.g., to measure temporal changes in the sample, they may be combined accumulatively to use a larger amount of the fluorophores present, or substantially all of the fluorophores, to image the structures in the sample and thus generate an optimal continuous image of the structure. It is also possible to determine relative displacements or drifts of structures with respect to each other from successively acquired individual images, compensate for these, and subsequently determine an image of the structure in an accumulative manner. It is further possible, if an image of structures has already been obtained in the sample or in partial areas of the sample in sufficiently good quality, to terminate the method altogether or to continue it only in those areas where the image quality is not yet satisfactory.

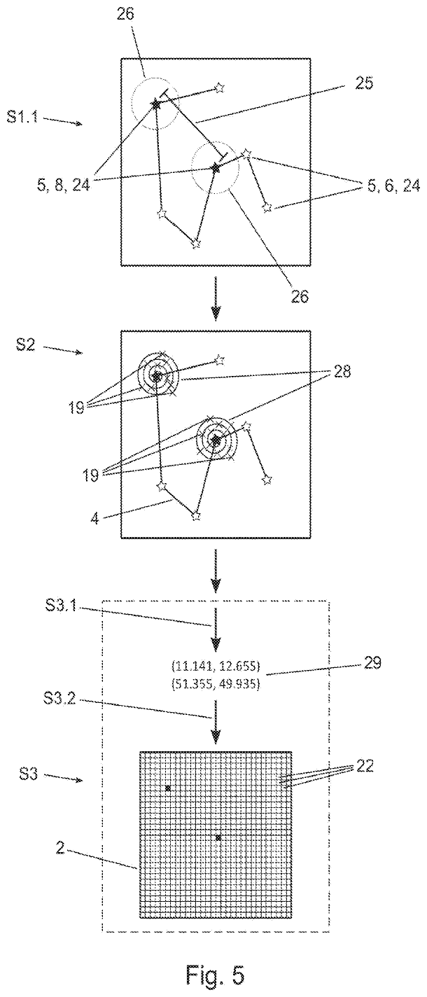

FIG. 5 shows a further preferred embodiment of a method according to the present disclosure, in which only individual, spatially separated molecules 24 of the fluorescent dye 5 are transferred from the protected, non-fluorescent form 6 to the activated, fluorescent form 8. The photoactivation step (not shown here) is thereby controlled so that the activated molecules 8, 24 of the fluorescent dye 5 have a distance 25 from each other which is not smaller than a minimum distance d. This minimum distance d is determined by the resolving power of the method used to pre-localize the activated fluorescent dye molecules 5, 8, 24 (see below) or, alternatively, by the degree to which the location of an activated fluorescent dye molecule is known based solely on knowledge of the location of activation and the resolving power achieved during scanning with excitation light 16 and fluorescence inhibition light 18, and must not be selected smaller than either of the associated two values.

Unlike in the method variant shown in FIG. 3, the photon numbers or intensities of fluorescent light obtained in the scanning and detection step S2 are not used here for the direct generation of an image 2 in the sense of a direct spatial representation of the detected brightness values, but first for the precise localization of the individual photoactivated molecules 8, 24 of the fluorescent dye 5 in a localization sub step S3.1. For this purpose, the scanning positions 19 are not arranged globally on a regular grid, but in groups of initially at least three scanning positions 19 around each photoactivated fluorescent dye molecule 5, 8, 24. Location estimates 26 of the photoactivated fluorescent dye molecules 5, 8, 24 required for this purpose may be known from the activation itself or may be obtained by fluorescence microscopy methods known to those skilled in the art and are not shown in detail here. In the simplest case, this can be done, e.g., by taking an epifluorescence image; alternatively, the sample may be scanned with the excitation light 16 to find individual activated dye molecules 5, 8, 24 in the sample and to make an initial location estimate 26.

In the subsequent scanning and detection step S2, the activated dye molecules 5, 8, 24 are illuminated with the intensity distributions of excitation light and fluorescence inhibition light starting with the respective at least three scanning positions 19 per molecule 24, and a photon number or intensity of fluorescence light is detected at each of the scanning positions 19. From the fluorescence detected at each of the at least three scanning positions 19, improved position estimates of the activated dye molecules 5, 8, 24 are now calculated in a triangulation fashion relative to the initial location estimates 26. For clarity, a scanning position at the location of the initial location estimate 26 is not shown. Such a scanning position can be used to reliably avoid ambiguities in the improved estimates of the locations of the activated dye molecules 5, 8, 24. Based on these improved location estimates, additional scanning positions 19 may now be determined for each activated dye molecule 5, 8, 24 and scanning may continue with increased maximum intensity of fluorescence inhibition light. Due to the fact that the scanning positions 19 are increasingly closer to the actual locations of the activated dye molecules 5, 8, 24, the scanning positions are then approximately on spiral paths 28. The scanning and detection step S2 is terminated after a predefined number of scanning points 19 per activated dye molecule 5, 8, 24 or when the location estimates fall below a maximum accepted error.

To generate the image 2 within the image generation step S3, first in a localization sub step S3.1 final coordinates 29 of the activated dye molecules 5, 8, 24 are determined from the associated photon numbers or intensities of fluorescent light and the scanning positions 19. The coordinates 29 of the localized molecules 24 are then displayed in a display sub step S3.2, e.g., in the form of a two-dimensional histogram with a suitable ruling. In order to obtain a high-resolution image of the structure in the sample in this way, the method steps (including photoactivation) are to be repeated (not shown) until the histogram comprises coordinates 29 of so many localized dye molecules 5, 24 that the structure 4 labeled with dye 5 is represented throughout by localized dye molecules 5, 24.

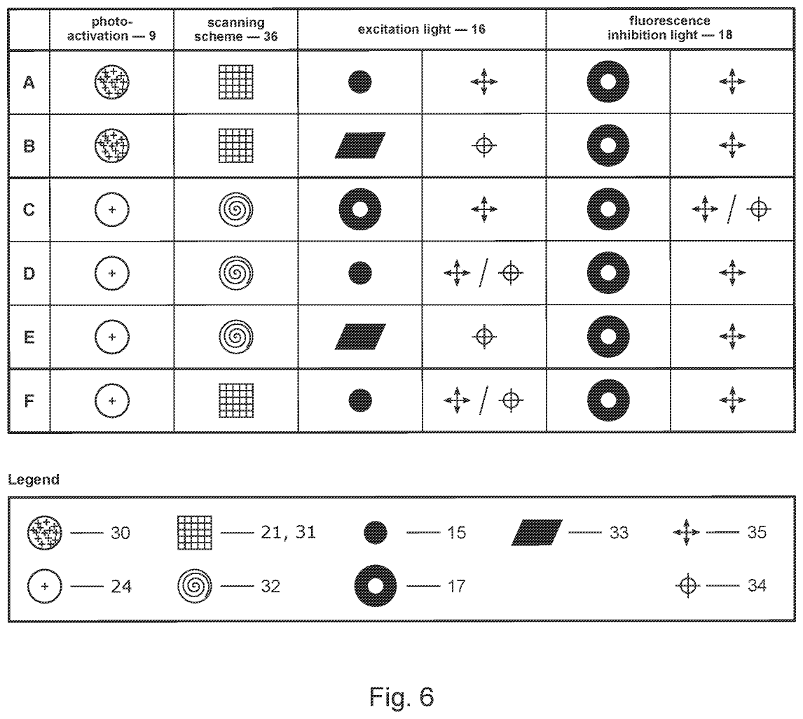

In FIG. 6, six different embodiments A to F of the methods according to the present disclosure are listed in tabular form. The list is exemplary and does not represent a conclusive list of all embodiments of the methods according to the present disclosure.

The embodiments shown have in common that the fluorescent dye is initially present in a protected, non-fluorescent form and that a portion of the fluorescent dye is converted to the activated, fluorescent form by illumination with activation light in a reaction comprising at least two light-induced reaction steps. In the table, the features distinguishing the embodiments shown are shown symbolically. In the second column of the table, it is indicated whether photoactivation 9 of single molecules 24 or of molecular ensembles 30, i.e., multiple molecules within a detection volume, is provided in the respective embodiment. In the third column of the table, the scanning scheme 36 is indicated, which specifies whether in the respective embodiment a regular scanning 31, in particular along a regular grid 21, or an adaptive scanning 32 is performed, in which the scanning positions are determined taking into account the fluorescence signals detected in previous scanning steps. In the fourth column, the intensity distribution of the excitation light 16 is shown symbolically, where a distinction is made between an intensity distribution 15 having a central intensity maximum, a homogeneous intensity distribution 33, and an intensity distribution 17 having a central intensity minimum. The fifth column shows whether scanning 35 is performed with the intensity distribution of the excitation light 16 or whether the intensity distribution of the excitation light 16 assumes a stationary position 34. The sixth and seventh columns reflect the corresponding features for the fluorescence inhibition light 18.

The particularly preferred embodiment shown in row A corresponds to the combination of focused excitation light 16 with an annular intensity distribution 17 of fluorescence inhibition light 18 or stimulation light, as known from STED microscopy. Both intensity distributions are scanned together and synchronously along a regular, usually Cartesian grid over the sample or a section of the sample. To generate a raster image, the fluorescence detected at each scan point is associated with a corresponding image pixel as a brightness value. The variant shown in line B differs from embodiment A in that the excitation light 16 is irradiated in the form of an intensity distribution 33 that is homogeneous in the scanning area. Scanning 35 is performed only with the intensity distribution 17 of fluorescence inhibition light 18 having the intensity minimum. Due to the illumination of the entire sample (or at least the section of the sample of interest) with excitation light 16, increased contributions of fluorescence light from areas outside the current scanning position occur in embodiment form B, which is why this embodiment form is less preferred. In embodiments C to F, photoactivation 9 takes the form of individual, spatially separated molecules 24, so that fluorescence from only one activated dye molecule at a time is detected when the sample or section of the sample is scanned. In the particularly preferred embodiment C, two intensity distributions 17, each having an intensity minimum, of excitation light and fluorescence inhibition light are superimposed, whereby the sample or the section of the sample is scanned at least with the excitation light 16. Insofar as the fluorescence inhibition light 18 remains at a stationary position 34 during the scanning of one activated dye molecule 24 at a time, the fluorescence inhibition light 18 serves quite predominantly only to suppress fluorescence contributions from regions outside the intensity minima; for this purpose, an intensity distribution with as broad an intensity minimum as possible is particularly advantageous. From the fluorescence signals detected at the scanning positions, previously determined position estimates of the activated dye molecules can be improved, i.e., the activated dye molecules can be localized with increasing accuracy. The scanning is preferably adaptive, i.e., the scanning points are determined taking into account the fluorescence signals detected at previous scanning positions, while simultaneously increasing the overall intensity of the excitation light 16. In this way, the localization of the dye molecules can be performed with an uncertainty of a few nanometers. In embodiment C, the scanning may optionally include excitation light 16 and fluorescence inhibition light 18 together; in this case, the fluorescence emission at the scanning positions of excitation and fluorescence inhibition light is modulated, which may complicate the calculation of improved position estimates. Embodiments D and E differ from embodiment C in that the fluorescence inhibition light 18 is used here not only to suppress fluorescence contributions from regions outside the intensity minima, but the fluorescence inhibition light 18 is used primarily to scan the sample or section of the sample at the scanning positions and modulates the fluorescence emission from the scanned dye molecule 24 to calculate improved position estimates. In these embodiments, the excitation light may be homogeneously distributed or patterned, for example in the form of a Gaussian focus', and may be stationary or displaced along with the fluorescence inhibition light 18 during scanning.

Finally, embodiment F combines features of embodiments A on the one hand and C to E on the other. Again, individual, spatially isolated dye molecules are activated, but the scanning 35 of these individual molecules is performed as in embodiment form A, i.e., with superimposed intensity distributions of excitation light 16 (with intensity maximum) and fluorescence inhibition light 18 (with intensity minimum), the scanning 35 being performed along a regular grid 21. Due to the photoactivation 9 of isolated dye molecules 24, a position estimate of the individual molecules 24 can be made from the photon numbers or intensities of the fluorescent light detected at the scanning positions along the grid 21 with an uncertainty well below that of the spacing of the scanning positions.

### LIST OF REFERENCE SIGNS

S1 Photoactivation step
S1.1 Pre-localization sub step
S2 Scanning and detection step
S2.1 Positioning sub step
S2.2 Illumination and detection sub step
S3 Image generation step
S3.1 Localization sub step
S3.2 Display sub step
1 Data memory
2 Image
3 Section
4 Structure
5 Fluorescent dye
6 Protected, non-fluorescent form
7 Activation light
8 Activated fluorescent form
9 Photoactivation
10 Two-step reaction
11 Reaction step
12 Cleavage
13 Photolabile protecting group
14 Photolabile protecting group
15 Intensity distribution with local maximum
16 Excitation light
17 Intensity distribution with local minimum
18 Fluorescence inhibition light
19 Scanning position
20 Grid point
21 Grid
22 Image pixel
23 Brightness value
24 (Single) molecule
25 Distance
26 Position estimate
28 Spiral path
29 Coordinates
30 Molecular ensemble
31 Regular scanning
32 Adaptive scanning
33 Homogeneous intensity distribution
34 Stationary position
35 Scanning
36 Scanning scheme

What is claimed is:

1. A method for localizing single molecules of a fluorescent dye in a sample, comprising the method step of
selecting a fluorescent dye that is convertible from a protected, non-fluorescent form to an activated, fluorescent form by illumination with an activation light,
and a group of method steps comprising the steps of
photoactivation of one or more molecules of the fluorescent dye, which are spaced apart by a minimum distance d from each other, from the protected, non-fluorescent form into the activated form by illumination with activation light,
forming an intensity distribution of an excitation light and an intensity distribution of a fluorescence inhibition light in the sample, wherein the intensity distribution of the fluorescence inhibition light comprises a local intensity minimum,
scanning the sample or a section of the sample with the intensity distribution of the fluorescence inhibition light comprising an intensity minimum at a sequence of scanning positions which are spaced apart from one another by a distance of not more than d/2;
detecting a photon number or an intensity of fluorescent light at each scanning position of the sequence, and associating the photon number or the intensity with the respective scanning position,
localizing activated dye molecules from the associated photon numbers or intensities of the fluorescent light and the scanning positions with an uncertainty of at most d/10 in at least one spatial direction,
wherein the value of d is such that the detected fluorescent light at each scanning position originates from only a single activated molecule of the fluorescent dye, and
wherein the fluorescent dye is selected such that the photoactivation comprises at least two respective light-induced reaction steps.

2. The method according to claim 1, wherein d≥250 nm.

3. The method according to claim 1, wherein the intensity distribution of the excitation light comprises a local intensity maximum, wherein the intensity distributions of the fluorescence inhibition light and the excitation light are substantially complementary to each other.

4. The method according to claim 1, wherein the scanning positions are arranged on a regular grid.

5. The method according to claim 1, the group of method steps is carried out repeatedly, wherein between the repetitions the respective activated dye molecules are converted into a non-fluorescent state.

6. The method according to claim 5, wherein a spatially high-resolution image of a structure in the sample is reconstructed from the locations of the activated dye molecules determined by the localization.

7. The method according to claim 1, wherein the activation light is used to form a plurality of illumination points in the sample.

8. The method according to claim 7, wherein the illumination points are arranged on a regular grid.

9. The method according to claim 1, wherein a light-induced reaction step is induced by multiphoton absorption.

10. The method according to claim 1, wherein all light-induced reaction steps are induced with activation light of identical wavelength.

11. The method according to claim 1, wherein one of the light-induced reaction steps is induced with activation light of a different wavelength than another light-induced reaction step.

12. The method according to claim 1, wherein at least one of the light-induced reaction steps is a photolytic cleavage of a photolabile protecting group.

13. The method according to claim 12, wherein the photolabile protecting group is selected from the group (each unsubstituted or substituted): nitrobenzyl, nitrophenethyl, nitroindolinyl, dinitroindolinyl, nitroveratryl, arylcarbonylmethyl, alkylphenacyl, hydroxyphenacyl, benzoin, hydroxycinnamate, o-nitro-2-phenethyloxy carbonyl, nitroanilide, coumarinyl, aminocoumarinyl, methoxycoumarylmethyl, anthraquinone-2-ylmethoxycarbonyl, (2-naphthyl)methyl, (anthracene-9-yl)methyl, (pyren-1-yl)methyl, (perylen-3-yl)methyl, (phenanthren-9-yl)methyl, o-hydroxyarylmethyl, azide, borondipyrro methenyl.

14. The method according to claim 12, wherein the light-induced reaction steps are photolytic cleavage reactions of identical photolabile protecting groups.

15. The method according to claim 12, wherein the light-induced reaction steps are photolytic cleavage reactions of different photolabile protecting groups.

16. The method according to claim 1, wherein at least two of the light-induced reaction steps are steps of a tandem reaction.

17. The method according to claim 16, wherein one step of the tandem reaction is reversible.

18. A method for localizing single molecules of a fluorescent dye in a sample comprising the method step of
selecting a fluorescent dye that is convertible from a protected, non-fluorescent form to an activated, fluorescent form by illumination with an activation light,
a first group of method steps comprising the steps of
photoactivation of one or more molecules of the fluorescent dye, which are spaced apart by a minimum distance d from each other, from the protected, non-fluorescent form into the activated form by illumination with activation light,
determining initial position estimates of one or more activated dye molecules with an uncertainty of no more than d/2,
forming an intensity distribution of an excitation light and an intensity distribution of a fluorescence inhibition light in the sample, wherein at least the intensity distribution of the fluorescence inhibition light comprises a local intensity minimum, wherein also the intensity distribution of the excitation light may comprise an intensity minimum;
and a second group of method steps comprising the steps of
scanning the sample or a section of the sample with one of the intensity distributions comprising an intensity minimum at a sequence of scanning positions, the sequence containing subsets each comprising at least two scanning positions which are arranged at a distance of less than d/2 around the position estimate of an activated dye molecule associated with the subset,
detecting a photon number or an intensity of fluorescent light at each scanning position of the sequence, and associating the photon number or the intensity with the respective scanning position,
determining a new position estimate for each of the activated dye molecules associated with a subset from the associated photon counts or intensities of fluorescent light and the scanning positions,
wherein the value of d is such that the initial position estimates can be unambiguously associated with the activated fluorescent dye molecules and that the detected fluorescent light at each scanning position originates from only a single activated molecule of the fluorescent dye in each case, and
wherein the fluorescent dye is selected such that the photoactivation comprises at least two respective light-induced reaction steps.

19. A method for generating spatially high-resolution images of a structure in a sample comprising the method steps of
selecting a fluorescent dye that is convertible from a protected, non-fluorescent form to an activated, fluorescent form by illumination with an activation light,
labeling the structure with the fluorescent dye,
as well as the following method steps carried out once or repeatedly
photoactivation of a subset of the fluorescent dye from the protected, non-fluorescent form into the activated form by illumination with activation light,
forming an intensity distribution of an excitation light and an intensity distribution of a fluorescence inhibition light in the sample, wherein the intensity distribution of the fluorescence inhibition light comprises a local intensity minimum,
scanning the sample or a section of the sample with the intensity distribution of the fluorescence inhibition light comprising an intensity minimum at a sequence of scanning positions,
detecting a photon number or an intensity of fluorescent light at each scanning position and associating the photon number or the intensity to the respective scanning position,
generating a high-resolution raster image of the structure from the associated photon numbers or intensities of the fluorescent light and the scanning positions by associating with each image pixel of the raster image a brightness value that is a monotonic function of the photon number or intensity of the fluorescent light detected at the respective scanning position or a respective set of scanning positions,
wherein the fluorescent dye is selected such that the photoactivation comprises at least two respective light-induced reaction steps.

20. Use of a fluorescent dye in a method according to claim 1, wherein the fluorescent dye is convertible from a protected, non-fluorescent form to an activated, fluorescent form by illumination with an activating light, and in that the conversion of the dye to the fluorescent form comprises at least two respective light-induced reaction steps.
